(19) United States
(12) Patent Application Publication
(10) Pub. No.: US 2008/0242945 A1
(43) Pub. Date: Oct. 2, 2008

(54) Facility management system

(75) Inventors: Nicola Gugliotti, Watertown, CT (US); Harold Todd Tyler, Lincoln, NE (US)

Correspondence Address: PILLSBURY WINTHROP SHAW PITTMAN, LLP, P.O. BOX 10500, MCLEAN, VA, 22102, US

(73) Assignee: Stanley Security Solutions, Inc., Indianapolis, IN (US)

(21) Appl. No.: 11/729,848

(22) Filed: Mar. 30, 2007

(51) Int. Cl. A61B 5/00

(52) U.S. Cl. 600/300

(57) ABSTRACT

The invention provides systems and methods for a acquiring, manipulating, and monitoring data associated with one or more assisted living facilities. The invention includes utilizing one or more monitoring devices to gather data regarding at least one assisted living facility. The data is then transmitted across a network and saved in a database. An enterprise management application then operates on the data to manipulate and display the data according to parameters and instructions specified by one or more users. The data may be used by the one or more users to monitor and manage the one or more assisted living facilities.

### BACKGROUND OF THE INVENTION

1. Field of the Invention

The invention relates to methods and systems for monitoring and managing multiple aspects of assisted living facilities, particularly, utilizing multiple monitoring devices present at assisted living facilities to gather information for an enterprise management application.

2. Description of Related Art

Health care facilities, including assisted living facilities that cater to the elderly and disabled, face an arduous task of monitoring their day to day operations. Assisted living facilities are under particular pressure to maintain a consistently high standard of care. Aspects of assisted living facility management other than patient care, such as, assistance response time, employee time, security and access control, equipment upkeep, medication distribution, or other areas, may be relevant when a patient (or relative thereof) is choosing an assisted living facility. Furthermore, entities operating multiple assisted living facilities face still additional challenges when attempting to monitor and manage the various aspects of these multiple facilities.

Often times, managers or other personnel involved with operating multiple assisted living facilities may be remotely located from some or all of the assisted living facilities. As such, collection, aggregation, and analysis of data for basic management tasks, identifying various conditions, assistance response times, identifying underperforming facilities, identifying over-performing facilities, trend analysis, and/or other data can be unwieldy.

Accordingly, there is a need for systems and methods for monitoring multiple aspects of multiple assisted living facilities and providing data regarding those multiple aspects to various remote locations.

### BRIEF SUMMARY OF THE INVENTION

The present invention relates to systems and methods that enable management of multiple assisted living facilities regardless of the geographic distribution of the facilities and/or the location of facility management personnel. As such, one aspect of the invention provides a system for acquiring, manipulating, and/or monitoring data regarding one or more assisted living facilities according to an embodiment of the invention. The system also enables management of the facilities associated with the system via various communication methods.

In some embodiments, a management system according to the invention may comprise an enterprise management application, a database, and/or other elements. The system may communicate/interact with one or more assisted living facilities (including various monitoring devices and computer systems of those facilities), one or more users, and/or other entities via a network (e.g., the Internet and/or other computer network).

The enterprise management application may comprise an Internet web site, an intranet site, or other host site or computer application maintained on the system of the invention. The enterprise management application may include one or more software modules for gathering data from monitoring devices of assisted living facilities and their associated systems, transmitting the data across a network, receiving the data, examining/processing the data, storing the data in a database, receiving data manipulation instructions (e.g., report parameters, query parameters, alert parameters, task parameters, or other instructions), manipulating the data, enabling one or more interfaces for the receipt of data and/or instructions, enabling one or more interfaces for the presentation of data, enabling the production and transmittal of alerts or tasks, establishing and editing one or more user accounts, and/or for performing other features of functions of the invention.

As used herein, an assisted living facility may include a facility wherein medical care and/or permanent or temporary residence is provided for the elderly, the physically disabled, the mentally disabled, other those otherwise in need of a facility providing both medical care and residential services. In some embodiments, other health care services and facilities (e.g., hospitals, hospices, home health care service, EMT/EMS services) may by used in conjunction with the invention.

Assisted living facilities may include and/or interface with one or more monitoring devices. A monitoring device used by the system may include any device or system that that enables the gathering of information regarding an assisted living facility. In some embodiments, a monitoring device may include a device that is fixed at an assisted living facility. In some embodiments, a monitoring device may include a device or system that is not necessarily permanently present at an assisted living facility (e.g., devices carried or worn by patients or employees, who may leave the facility).

In many instances, as will be seen by the description herein, monitoring devices may include devices or systems that serve a separate function, but which gather, record, or pass data regarding performance of that separate function, thus monitoring the separate function. For example, in some embodiments, monitoring devices may include devices and systems involved in patient safety at an assisted living facility. These devices and systems may include, for example, medical alert pendants having transmitters therein that are typically worn by patients. The transmitter, when activated by the patient, may send a signal indicating that the patient requesting assistance. The pendant itself, and/or the associated system receiving the signal, may act as a monitoring device by the fact that information associated with the signal is logged and/or recorded and ultimately transmitted to, and stored in, a central management database.

Other patient-related devices or systems, such as, for example, blood pressure monitors, heart rate monitors, blood gas monitors, respiration monitors, temperature monitors, in-room distress devices (e.g., a button on a wall or piece of furniture, as opposed to mobile pendants), nurse call buttons, and/or other devices/systems may act as monitoring devices. Information from these devices and systems may be logged, recorded or otherwise captured, transmitted across a network, and stored in a management database.

In some embodiments, monitoring devices may include video cameras, monitors, and/or associated equipment. For example, in some embodiments, video cameras and monitors may be used to monitor patients and record and/or trigger an patient distress events. In some embodiments, personnel may monitor video feed and manually trigger a patient alert or other patient event. In other embodiments, video feeds may be automatically monitored and alerts/events may be automatically triggered.

Other patient safety related systems may similarly act as monitoring devices. For example, staff members of an assisted living facility may wear/carry pendants, pagers, phones, handheld computers, or other devices that transmit signals related to when medical personnel has arrived at a patient whose pendent has transmitted a request for assistance signal (or for whom such a signal has been otherwise initiated). When a patient distress signal is detected, medical staff may be dispatched to the patient's location. Upon reaching the patient, the dispatched personnel may utilize their response devices to transmit a signal to a system that indicates help has arrived at the patient. In this manner, response times or other information related to patient safety may be monitored and transmitted to a management database. As such, the staff devices and/or their associated systems (which may be the same systems associated with the patient pendants) may also be considered patient monitoring devices.

Devices and systems related to other aspects of assisted living facilities may considered monitoring devices. For example, in some embodiments, employee time logging systems, such as systems whereby employees of an assisted living facility indicate their time worked, may act as monitoring devices whereby data regarding employee time may be collected, recorded, or otherwise captured, transmitted across a network, and stored in a management database.

In some embodiments, security and access control devices and systems may act as monitoring devices. For example, entry and exit control systems, motion control sensors, equipment security systems (e.g., anti-theft systems), or other security systems may be utilized to gather data that is recorded, or otherwise captured, transmitted across a network, and stored in a management database for use by the management system. Alone or in conjunction with the above mentioned security systems, automatic door or other entry and exit control systems may be utilized as monitoring devices. Similarly, drug and medication logs and security systems may be utilized as monitoring devices for gathering data for the management system.

In some embodiments, equipment service records and systems for equipment servicing may be used as monitoring devices. For example, time between servicing, frequency of service, type of service, and/or other service related information may be collected, transmitted across a network, and stored in a management database.

Assisted living facilities may include computer-implemented devices and/or systems necessary to gather/receive data from various monitoring devices, store this data (at least temporarily), connect with network (e.g., modems, etc.), transmit data across a network to the management system, and/or perform other features or functions of the invention. These computer-implemented devices and systems may be or include one or more servers, desktop computers, laptop computers, handheld computers, personal digital assistants, cell phones, smart phones, modems, or other computer implemented devices.

Users of the management system may include managers, medical personnel, administrative personnel, other employees of assisted living facilities, or other parties with an interest in monitoring operation of one or more aspects of assisted living facilities (e.g., consultants, auditors, government entities, regulators, relatives of patients, or other parties) that may access the management system via a network.

Users may access the management system using one or more computer-implemented user devices. A user device may include a server, a desktop computer, a laptop computer, a dumb terminal, a handheld computer, a personal digital assistant, a cell phone, a smart phone, pager, or other computer-implemented device, whether wireless or not. Users may utilize a graphical user interface to interact and/or interface with the management system. In some embodiments, one or more modules of the enterprise management application (e.g., an interface module) may support graphical user interfaces or other interfaces of the invention.

The invention also includes a process for acquiring, manipulating, and monitoring data associated with assisted living facilities. In one embodiment, one or more event monitoring devices record or otherwise collect data regarding events and operations of one or more assisted living-facilities. As described above, the events and operations associated with an assisted living facility may include data regarding patients of facilities (including patient distress events or calls and response times to those events or calls), employee time, security systems, entry/exit control systems, drug or medication dispensing, equipment service, and/or other data.

The data associated with the events and/or operations of assisted living facilities may then be transmitted from the assisted living facilities, over a network (e.g., the Internet and/or other computer network), where it is received by an assisted living facility management system. In some embodiments, the data may be sent to the management system as it is collected by monitoring devices. In some embodiments, the data may be gathered and at least temporarily stored in one or more computer systems of an assisted living facility or other systems and sent to the management system at a later time.

In some embodiments, the data received at the management system from the monitoring devices may then be processed prior to storing the data in a system database. For example, the data may be checked for completeness, for obvious entry errors or data originating from malfunctioning monitoring devices, which may be flagged for follow-up or disposal, and/or for other purposes. The processed data may then be stored in a system database.

The stored data may then be queried, filtered, analyzed, and or otherwise manipulated for any number of uses. In some embodiments, the stored data may be utilized to construct one or more reports, alerts, tasks, and/or other documents/communications associated with one or more aspects of one or more assisted living communities. One or more modules (e.g., a query module, a report module, or other module) of the enterprise management application may enable querying, filtering, report generation, alert generation, task generation, and/or other manipulation of data stored in the system database.

The stored data may then be accessed by, or presented to, one or more users via one or more user devices. In some embodiments, one or more modules (e.g., an interface module) of the enterprise application may enable a portal accessible via a network, whereby one or more users may access data regarding one or more aspects of one or more assisted living facilities. In some embodiments, the portal may be a web portal accessible via the internet and may include multiple graphical user interfaces and/or other interfaces enabled by the enterprise management application. This web portal may enable a user to not only view the data but may enable a user to enter information and/or direct manipulation of the data.

For example, users, interacting via the portal or other interface of the invention, may specify report or query parameters for the generation of reports or query results. These parameters may then be applied to the data in the system database to generate a report or query result for presentation to users. The report or result may then be displayed to users in a graphical user interface.

As described above, users may access the management system and associated databases of the invention using mobile or wireless user devices. As such, one or more modules (e.g., an interface module) of the enterprise management application may enable the portal to present graphical user interfaces compatible with mobile devices.

In some embodiments, the enterprise management application may enable alerts and/or tasks to be sent to one or more users. For example, in some embodiments, alerts may be sent to high level business managers to keep them apprised of the operations of one or more assisted living facilities or for other purposes. In some embodiments, alerts may be sent to facility managers or health care staff on site at an assisted living facility to keep them apprised of conditions at the assisted living facility, to assign tasks to certain personnel, or for other purposes. In some embodiments, alerts and/or tasks may be sent to other users.

In some embodiments, the invention may include a computer/processor-readable medium that includes computer/processor-readable code that causes a processor, computer, or other computer system or part thereof to acquire and manipulate data associated with one or more assisted living facilities and otherwise enable monitoring and management of the one or more facilities. As such, the computer/processor readable code includes code for performing some or all of the features and functions described herein such as, for example, gathering data from monitoring devices and their associated systems, transmitting the data across a network, receiving the data, examining/processing the data, storing the data in a database, receiving data manipulation instructions (e.g., report parameters, query parameters, alert parameters, or other instructions), manipulating the data, enabling one or more interfaces for the receipt of data and/or instructions, enabling one or more interfaces for the presentation of data, enabling the production and transmittal of alerts or tasks, and/or for enabling other features or functions of the invention.

These and other objects, features, and advantages of the invention will be apparent through the detailed description and the drawings attached hereto. It is also to be understood that both the foregoing summary and the following detailed description are exemplary and not restrictive of the scope of the invention.

### BRIEF DESCRIPTION OF THE DRAWINGS

FIG. 1A illustrates an example of a system for acquiring, manipulating and monitoring data regarding one or more assisted living facilities according to various embodiments of the invention.

FIG. 1B illustrates an example of a medical alert pendant and its associated computer system, according to various embodiments of the invention.

FIG. 1C illustrates an example of a patient wearing a medical alert pendant according to various embodiments of the invention.

FIG. 2 illustrates an example of a process for acquiring, manipulating, and monitoring data regarding assisted living facilities according to various embodiments of the invention.

FIG. 3 illustrates an example of a graphical user interface according to various embodiments of the invention.

FIG. 4 illustrates an example of a graphical user interface according to various embodiments of the invention.

FIG. 5 illustrates an example of a graphical user interface according to various embodiments of the invention.

FIG. 6 illustrates an example of a graphical user interface according to various embodiments of the invention.

FIG. 7 illustrates an example of a graphical user interface according to various embodiments of the invention.

FIG. 8 illustrates an example of a graphical user interface according to various embodiments of the invention.

FIG. 9 illustrates an example of a graphical user interface according to various embodiments of the invention.

FIG. 10 illustrates an example of a graphical user interface according to various embodiments of the invention.

FIG. 11 illustrates an example of a graphical user interface according to various embodiments of the invention.

FIG. 12 illustrates an example of a graphical user interface according to various embodiments of the invention.

FIG. 13 illustrates an example of a graphical user interface according to various embodiments of the invention.

FIG. 14 illustrates an example of a graphical user interface according to various embodiments of the invention.

FIG. 15A illustrates an example of a mobile user device according to various embodiments of the invention.

FIG. 15B illustrates an example of a mobile device graphical user interface according to various embodiments of the invention.

FIG. 16A illustrates an example of a graphical user interface according to various embodiments of the invention.

FIG. 16B illustrates an example of a graphical user interface according to various embodiments of the invention.

FIG. 16C illustrates an example of a graphical user interface and an alert according to various embodiments of the invention.

### DETAILED DESCRIPTION OF THE INVENTION

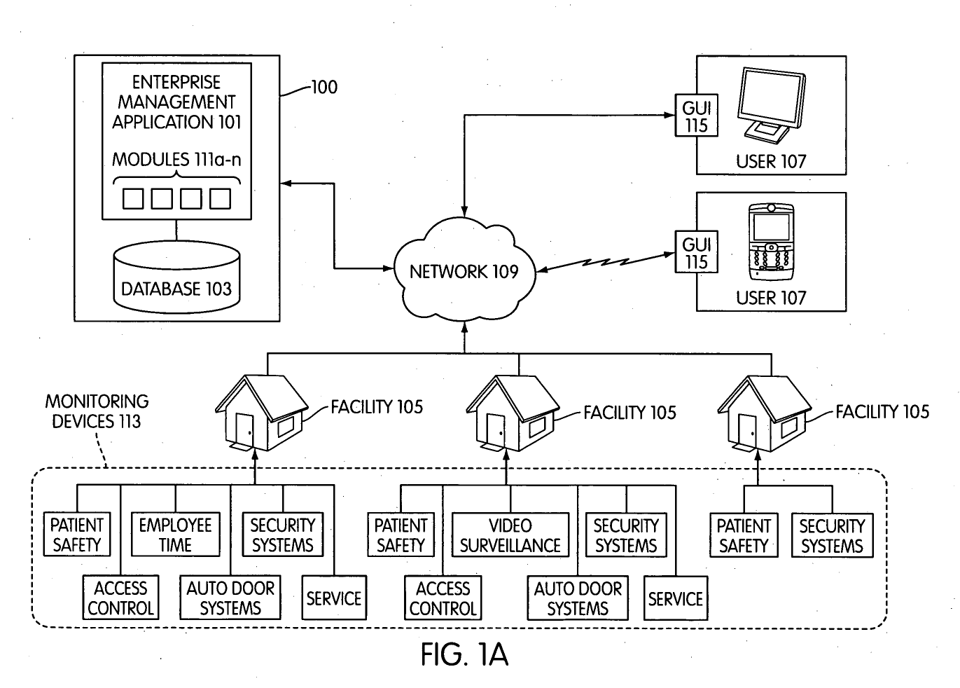

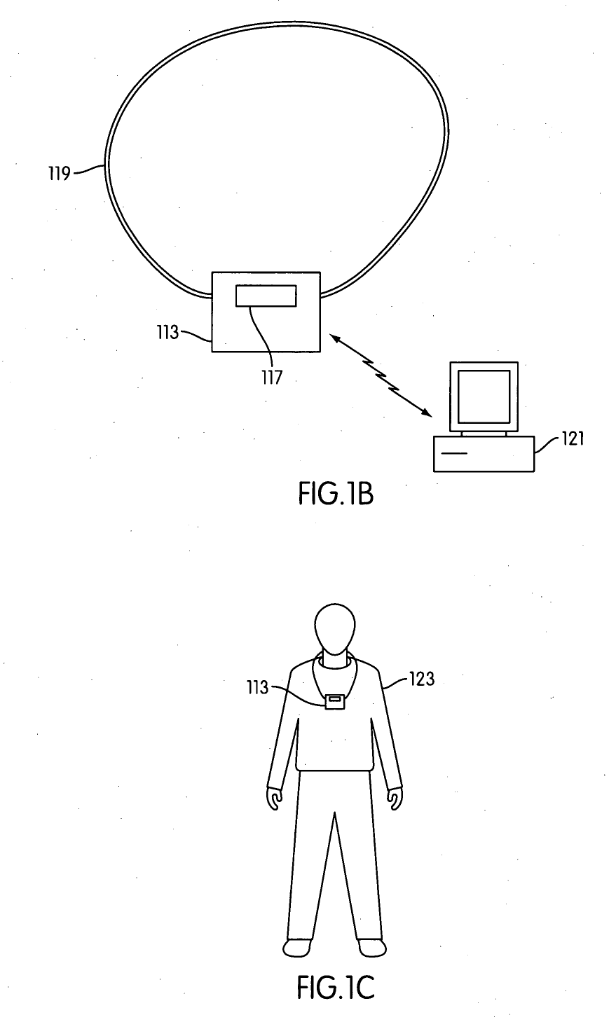

FIG. 1A illustrates an example of a system 100 for acquiring, manipulating, and monitoring data regarding one or more assisted living facilities according to an embodiment of the invention. System 100 may comprise an enterprise management application 101, a database 103, and/or other elements. System 100 may communicate/interact with one or more assisted living facilities 105, one or more users 107, and/or other entities via network 109.

Enterprise management application 101 may comprise an Internet web site, an intranet site, or other host site or computer application maintained on system 100. Accordingly, one or more hardware devices such as, for example, processors, servers, desktop computers, memory devices, or other devices or elements, may be included in system 100 to support enterprise management application 101, database 103, and/or other features and functions of the invention.

Enterprise management application 101 may include one or more software modules 111a-n for gathering data associated with one or more assisted living facilities 105 from monitoring devices and their associated systems, transmitting the data across a network, receiving the data, examining/processing the data, storing the data in a database, receiving data manipulation instructions (e.g., report parameters, query parameters, alert parameters, task parameters, or other instructions), manipulating the data, enabling one or more interfaces for the receipt of data and/or instructions, enabling one or more interfaces for the presentation of data, enabling the production and transmittal of alerts or tasks, establishing and editing one or more user accounts, and/or for performing other features or functions of the invention.

In particular, enterprise management application 101 may include an interface module for supporting graphical user interfaces (used for the receipt of data/instructions from users as well as the presentation of data or other interaction with system 100) and other interfaces, such as those between monitoring devices or their associated systems and system 100; a query module that enables querying of data in database 103; a report module that enables the generation of reports or other data displayed using the data from database 103, an alert module that enables the generation of alerts or tasks using data in database 103; a user account module that enables the setup and editing of one or more user accounts and associated account information; and/or other modules for performing any of the features or functions of the inventions described herein. One or more of the modules 111a-n comprising control application 101 may be combined. For some purposes, not all modules may be necessary.

According to an embodiment of the invention, database 103 may be, include, or interface to, for example, an Oracle™ relational database sold commercially by Oracle Corporation. Other databases, such as lnformix™, DB2 (Database 2) or other data storage or query formats, platforms, or resources such as OLAP (On Line Analytical Processing), SQL (Standard Language Query), a SAN (storage area network), Microsoft Access™ or others may also be used, incorporated, or accessed by the invention. Database 103 may include any combination of databases or other data storage devices, and may receive and store information regarding one or more assisted living facilities, report parameters, alert preferences, task parameters, user accounts, and/or other information.

In some embodiments, enterprise management application 101 and its associated devices (e.g., servers, processors, memory, etc.) may be considered centrally located and as such may provide a central station for assisted living facility data acquisition and manipulation and for monitoring and management of geographically distributed assisted living facilities. In some embodiments, enterprise management application and its associated modules and devices may be distributed across a plurality of devices and/or locations, while still providing the features and functions of the invention.

An assisted living facility 105, may include a facility wherein medical care and/or permanent or temporary residence is provided for the elderly, the physically disabled, the mentally disabled, or those otherwise in need of a facility providing both medical care and residential services. In some embodiments, other health care services and facilities (e.g., hospitals, hospices, home health care service, EMT/EMS services) may by used in conjunction with the invention. In some embodiments, one or more assisted living facilities 105 may be geographically distributed from one another. In some embodiments, the geographical distribution may include facilities that are located in different neighborhoods, towns, cities, counties, provinces, states, countries, or otherwise geographically distributed.

Assisted living facilities 105 may include and/or interface with one or more monitoring devices 113. Monitoring devices 113 may include any device or system that that enables the gathering of information associated with an assisted living facility. In some embodiments, monitoring devices 113 may include a device that is present at an assisted living facility 105. In some embodiments, monitoring devices 113 may include devices not necessarily perpetually present at an assisted living facility 105 (e.g., such as, for example, devices worn or carried by a patient or employee).

In many instances, as will be seen by the description herein, monitoring devices 113 may include devices or systems that serve a separate function, but which gather, record, or pass data regarding performance of that separate function to system 100, thus monitoring the separate function. For example, in some embodiments, monitoring devices 113 may include devices and systems involved in patient health or safety at an assisted living facility. These devices and systems may include, for example, medical alert pendants having transmitters therein that are typically worn by patients. The transmitter, when activated by the patient, may send an alert signal indicating that the patient is in distress or otherwise indicating an event. The pendant itself, and/or the corresponding system receiving the distress signal (e.g., computer system 121 of FIG. 1B), may act as a monitoring device 113 by the fact that information associated with the distress signal is logged and or recorded and ultimately stored in database 103. Information associated with the distress signal may include, for example, the fact that the signal was sent, what pendent or other monitoring device 113 it was sent by, who (e.g., what patient) is associated with that pendent or other monitoring device 113, what apartment or room in an assisted living facility is associated with the pendant or other monitoring device 113 (the monitoring device sending the signal may be associated with a particular patient residence apartment, or may be associated with a common area, medical treatment area, or other room or area), what type of alert is associated with the signal, what assisted living facility the pendant or other monitoring device 113 is associated with, when the signal was sent, or other information associated with the distress signal. In some embodiments, this information may be initially stored by the patient monitoring system associated with the pendent (e.g., system 121 of FIG. 1B) or another system local to the assisted living facility 113 wherein the patient monitoring system resides for later transmittal via network 109 to database 103. In some embodiments, the information associated with the distress signal may be transmitted via network 109 for initial storage in database 103.

FIGS. 1B and 1C illustrate a medical alert pendent 113, which is an example of a monitoring device 113 according to some embodiments of the invention. In some embodiments, as illustrated in FIG. 1B, a medical alert pendent 113 may include an activation button 117, by which a transmitter (not illustrated) of pendant 113 may be activated/instructed to send an alert signal to associated computer system 121. In some embodiments, the pendant may include a strap 119, which may aid in a patient 123 wearing the pendant, for example, around the neck, as illustrated in FIG. 1C. Other types of medical alert devices and/or other monitoring devices 113 may be used with the invention.

Other patient-related devices or systems, such as, for example, blood pressure monitors, heart rate monitors, blood gas monitors, respiration monitors, temperature monitors, in-room distress devices (as opposed to mobile pendants), nurse call buttons, and/or other devices or systems may act as monitoring devices 113. Information from these devices and systems may be logged, recorded or otherwise captured, transmitted across network 109, and stored in database 103 for use by system 100.

In some embodiments, monitoring devices 113 may include video cameras, monitors, and/or associated equipment. For example, in some embodiments, video equipment may be used to monitor patients and record and/or trigger an patient distress events. In some embodiments, video equipment may be used to monitor other assisted living facility operations. In some embodiments, assisted living facility personnel may monitor video feeds/footage and manually trigger a patient alert or other patient event. In other embodiments, video feeds/footage may be automatically monitored and/or alerts/events may be automatically triggered.

Other patient information related systems may similarly act as monitoring devices 113. For example, staff members of an assisted living facility 105 may wear/carry pendants, pagers, phones, handheld computers, or other devices that transmit response signals related to when medical personnel has arrived at a patient whose pendent or other monitoring device 113 has transmitted a request for assistance signal. For example, when a request for assistance signal or other signal indicative of an event is detected from a patient pendent or other monitoring device 113, medical staff may be dispatched to the patient's location. Upon reaching the patient, the dispatched staff member(s) may utilize their staff member device to transmit a signal to a system that indicates help has arrived at the patient (e.g., a dispatched staff member may actuate a “response switch” that causes a monitoring device 113 to generate a response signal indicating the patient event has been attended to). In this manner, response times (e.g., elapsed time between the distress event signal and the response signal) or other information related to patient safety may be monitored and transmitted to database 103. As such, the staff devices and/or their associated systems (which may be the same systems associated with the patient pendants) may also be considered patient monitoring devices 113.

Devices and systems related to other aspects of assisted living facilities 105 may be considered monitoring devices 113. For example, in some embodiments, employee time logging systems, such as systems whereby employees of an assisted living facility indicate their time worked, may act as monitoring devices 113, whereby data regarding employee time may be collected, recorded, or otherwise captured, transmitted across network 109, and stored in database 103 for use by system 100.

In some embodiments, security and access control devices and systems may act as monitoring devices. For example, access control systems, motion sensors, equipment security systems (e.g., anti-theft systems), or other security systems may be utilized to gather data that is recorded or otherwise captured, transmitted across network 109, and stored in database 103 for use by system 100. Alone or in conjunction with the above mentioned security systems, automatic door or other entry and exit control systems may be utilized as monitoring devices 113. Similarly, drug and medication logs and associated security systems may be utilized as monitoring devices 113 for gathering data for system 100.

In some embodiments, service records and systems for equipment service recordation may be used as monitoring devices 113. For example, time between servicing, frequency of service, information regarding specific service calls (e.g., type of service, parts replaced or operated on, service person's identity or company affiliation), or other service related information may be collected, transmitted across network 109, and stored in database 103.

In some embodiments, inventory records, supply purchasing records, supply disposal or usage records, and/or other supply-related records may be used as or in conjunction with monitoring devices 113. For example, records and record keeping systems (e.g., associated computer systems, databases, paper records) related to inventory levels, purchasing of new supplies or equipment, disposal of supplies or equipment or other supply related records may be utilized to gather information related to assisted living facilities 105.

Manual systems and devices may be used as monitoring devices 113. For example, non-computer-implemented systems and devices may be used to manually collect data that is ultimately entered in to electronic or digital format and transmitted to database 103 across network 109 for use in system 100. As such, manual implements, such as written log sheets, sign-in/sign-out sheets, punch card systems, and/or other manual implements and elements may be utilized as part of monitoring devices 113. Other systems and devices may be used as monitoring device 113 to gather other types of information for use in system 100 or otherwise used as part of the invention.

Assisted living facilities 105 may include computer-implemented devices and/or systems necessary to gather/receive data from various monitoring devices 113, store this data (at least temporarily), connect with network 109 (e.g., modems, etc.), transmit data across network 109 to database 103, and/or perform other features or functions. These computer-implemented devices and systems may be or include one or more servers, desktop computers, laptop computers, dumb terminals, handheld computers, personal digital assistants, cell phones, smart phones, modems, or other computer-implemented devices.

It should be noted that the term assisted living facility as used herein may be analogous to the term community as used in the disclosure and one or more of the figures.

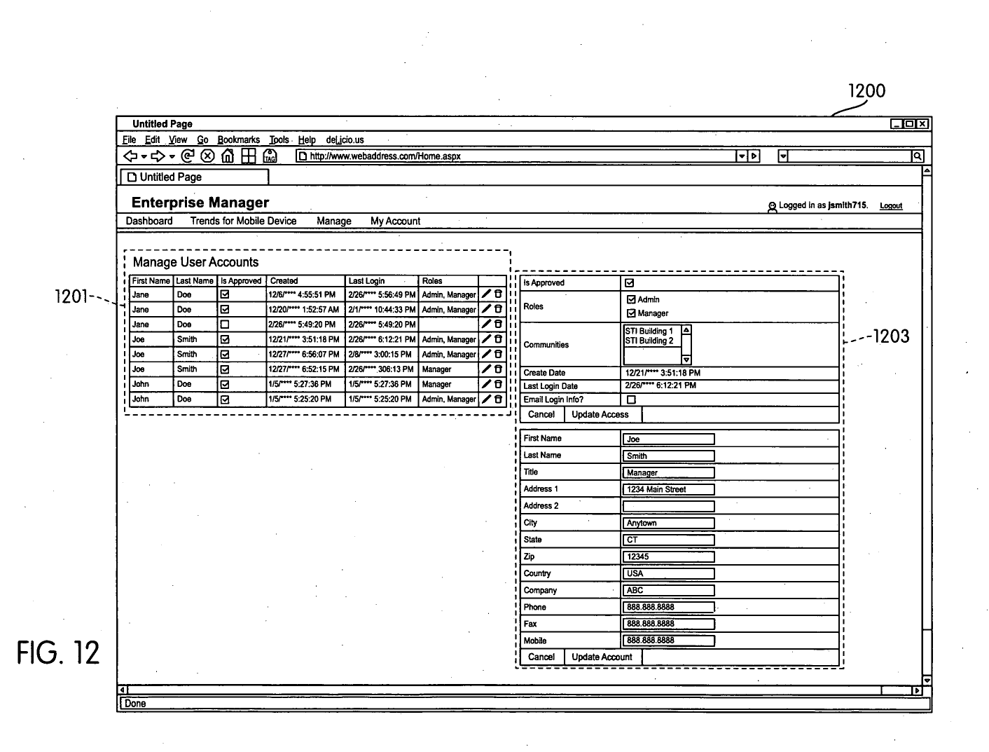

Users 107 may include managers, medical personnel, administrative personnel, other employees of assisted living facilities 105, or other parties with an interest in monitoring operation of one or more aspects of assisted living facilities (e.g., consultants, auditors, government entities/regulators, relatives of patients, or other parties) that may access system 100 via network 109. In some embodiments, those with access to system 100 may be restricted, depending on the intended use of system 100. For example, in some embodiments, only management personnel of an assisted living facility 105 may have access system 100. In some embodiments, access may be more permissive. In some embodiments, access for certain users may be limited on a per-facility basis or otherwise segmented or restricted. As discussed below, system 100 may provide an interface (see e.g., FIG. 12) that enables certain users (e.g., high level managers) to define and/or edit the access rights of other users 107.

Users 107 may access system 100 using one or more computer-implemented user devices. A user device may include a server, a desktop computer, a laptop computer, a dumb terminal, a handheld computer, a personal digital assistant, a cell phone, a smart phone, pager, or other computer-implemented device, whether wireless or not. Users 107 may utilize a graphical user interface 115 (GUI 115) to interact and/or interface with system 100. In some embodiments, one or more modules 111a-n (e.g. an interface module) may support GUI 115.

Network 109 may include any one or more of, for instance, the Internet, an intranet, a PAN (Personal Area Network), a LAN (Local Area Network), a WAN (Wide Area Network), a SAN (Storage Area Network), or a MAN (Metropolitan Area Network). Any suitable communications link may be utilized to connect elements of the invention to one another via network 109, including any one or more of, for instance, a copper telephone line, a Digital Subscriber Line (DSL) connection, a Digital Data Service (DDS) connection, an Ethernet connection, an Integrated Services Digital Network (ISDN) line, an analog modem connection, a cable modem connection, wireless connection (including wireless “hot-spots”), or other connection.

System 100 and other components of FIG. 1 are illustrated in an example configuration. Other configurations may exist. Those having skill in the art will appreciate that the invention described herein may work with various configurations. Accordingly, more or less of the aforementioned components may be used and/or combined in various embodiments. It should also be understood that various software modules 111a-n, enterprise management application 101, and database 103, that are utilized to accomplish the functionalities described herein may be maintained on one or more of user devices, assisted living facility devices, management devices, or other components as necessary. In other embodiments, as would be appreciated, the functionalities described herein may be implemented in various combinations of hardware and/or firmware, in addition to, or instead of, software.

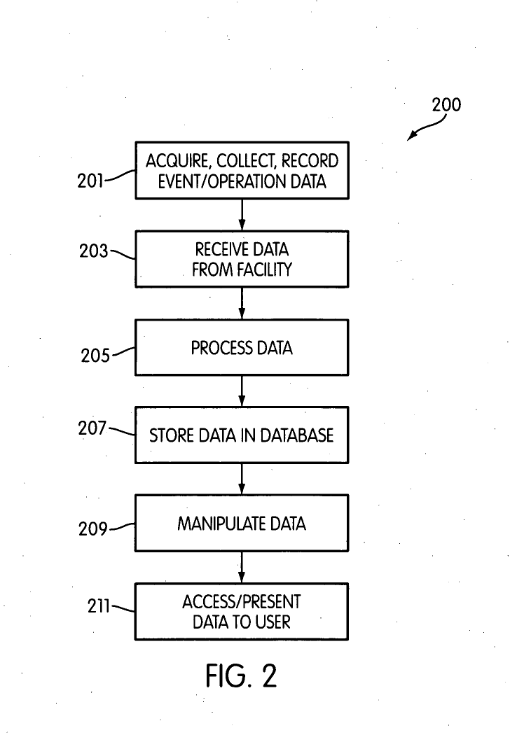

FIG. 2 illustrates a process 200, which is an example of a process for acquiring, manipulating, and monitoring data regarding assisted living facilities. Process 200 includes an operation 201, wherein one or more monitoring devices 113 record or otherwise collect data associated with events and operations of one or more assisted living facilities 105. As described above, the events and operations associated with an assisted living facility may include data regarding patients of facilities (including patient distress events or calls and response times to those events or calls), employee time, security systems, entry/exit control systems, drug or medication dispensing, equipment service records, supply or equipment inventory records, supply or equipment purchase records, supply or equipment disposal records, or other data.

In an operation 203, the data associated with the events and/or operations of assisted living facilities 105 is transmitted from the assisted living facilities 105, over network 109, where it is received by system 100. In some embodiments, the data may be sent to system 100 as it is collected by monitoring devices 113. In some embodiments, the data may be gathered and at least temporarily stored in one or more computer systems of an assisted living facility 105 or other systems and sent to system 100 at a later time. In some embodiments, the transmittal of data from computer systems of assisted living facilities 105 to system 100 may occur on a predetermined interval or may occur on demand from users 107 or administrators of system 100. In some embodiments, the data associated with events/operation of assisted living facilities 105 may be retained on the computer systems of assisted living facilities 105 after it has been sent to system 100.

In some embodiments, one or more modules 111a-n (e.g., interface module) of enterprise management application 101 may provide or support the interfaces necessary for the various devices and/or systems transmitting assisted living facility data across network 109 to system 100.

In an operation 205, the data received from the monitoring devices 113 may be processed. In some embodiments, one or more modules 111a-n of enterprise management application 101 may perform one or more processing operations prior to storing the data in database 103. For example, the data may be checked for completeness, for obvious entry errors or data originating from malfunctioning monitoring devices 113, which may be flagged for follow-up or disposal (e.g., if the average number of patient events per day equals 10 and a particular monitoring device 113 indicates that patient X had 10,000 events in one day, this may indicate an error in the data), and/or for other purposes. The processed data may be stored in database 103 in an operation 207.

In an operation 209, enterprise management application 101 may enable the stored data to be queried, filtered, analyzed, mined, and/or otherwise manipulated for any number of uses. In some embodiments, the stored data may be utilized to construct one or more reports, alerts, tasks, or other documents/communications regarding one or more aspects of one or more assisted living communities 105. One or more modules 111a-n (e.g., a query module, a report module, an alert module, or other module) of enterprise management application 101 may enable querying, filtering, report generation, alert generation, task generation, and/or other manipulation of data stored in database 103.

In an operation 211, the stored data may be accessed by or presented to one or more users 107. In some embodiments, one or more of modules 111a-n (e.g., an interface module) of enterprise management application 101 may enable a portal accessible via network 109, whereby one or more users may access data regarding one or more aspects of one or more assisted living facilities and/or input data to system 100. In some embodiments, the portal may be a web portal accessible via the internet (network 109) and may include GUI 115 and/or other interfaces enabled by enterprise management application 101. This web portal may enable a user 107 to not only view the data but may enable a user 107 to direct the manipulation of the data.

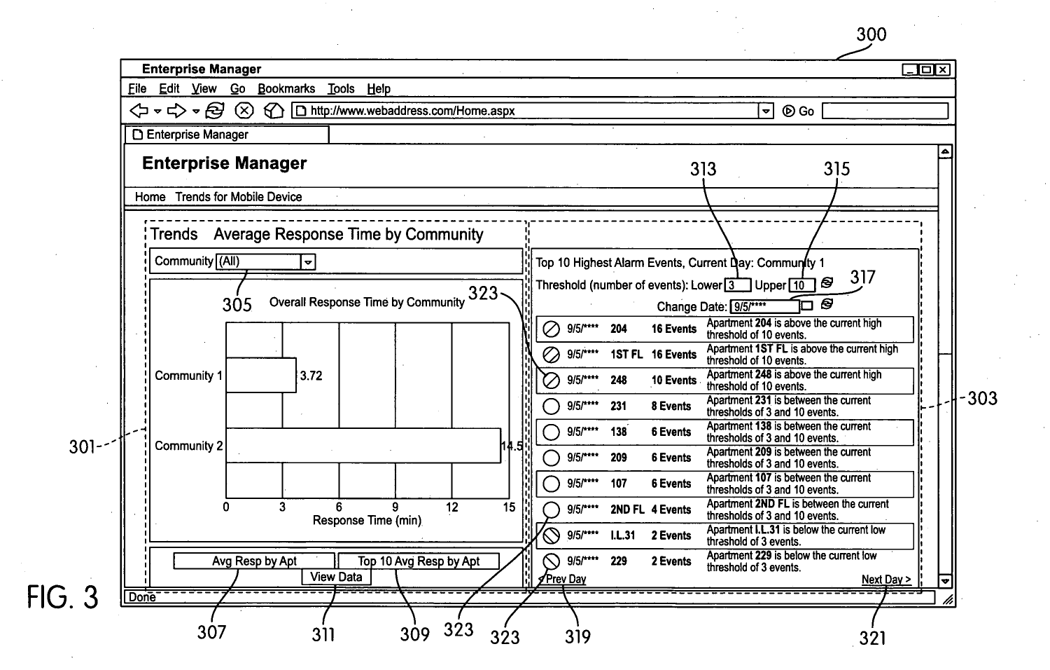

For example, users 107, interacting via the portal or other interface of the invention, may specify report parameters for the generation of reports. These report parameters may then be applied to the data in database 103 by, for example, a report module, to generate a report for presentation to users 107. The report may then be displayed to users 107 in a graphical user interface supported by an interface module. FIG. 3 illustrates an interface 300, which is an example of a graphical user interface according to an embodiment of the invention. Interface 300 is an example of a report generated from specified report parameters applied to a database of assisted living facility data.

To arrive at interface 300, a user 107 may connect his or her user device to network 109, access the portal provided by enterprise management application 101, and enter certain report parameters. In some embodiments, the portal may be a secure portal. As such, upon initially accessing the portal, a user 107 may be required to provide a username and/or password or comply with some other authentication/authorization scheme. A user account module of enterprise management application 101 may support one or more user accounts in database 103, each of which may include authorization information (e.g., user name and/or password) and/or other information associated with individual users 107. After complying with the authorization scheme, the user may be brought to a “home” interface (e.g., homepage) from which the user may navigate through the many features provided by the portal. In some embodiments, the user may be presented with one or more options for viewing/manipulating assisted living facility data such as, for example, browse data in database 103, browsing existing reports or automatically generated reports, enter report parameters, enter query parameters, entering alert parameters/preferences, and/or otherwise view data or direct data manipulation. These options may include any number of drop down lists, menus, check boxes, buttons, radio buttons, hyperlinks, scroll bars, text input devices, or other input regions/devices.

Interface 300 is a report that includes a graph depicting overall patient distress event response time across multiple assisted living facilities 105 (community 1 and community 2) in a section 301 and a table depicting the top 10 facility alarm events for community 1 in a certain day, by apartment, in a section 303. As mentioned above, to arrive at interface 300, a user 107 may have previously navigated the portal or other interface provided by system 100 to input report parameters selecting the parameters necessary to arrive at the reports given in sections 301 and 303 or otherwise select these reports. In some embodiments, these reports may be pre-generated and a user 107 need only select from a list of pre-generated reports.

Interface 300 also illustrates certain features whereby users 107 may further direct data manipulation and report generation such as, for example: drop down menu 305, wherein the communities included in section 301 may be selected; buttons 307 and 309, wherein display of different data reports may be selected; button 311 wherein the underlying data of the graph of section 301 may be viewed; text input boxes 313 and 315, wherein upper and lower thresholds for patient distress events displayed in section 303 may be input; text input box 317, wherein the date for the patient distress event data displayed in section 303 may be input; hyperlinks 319 and 321 to toggle through dates for section 303, and differential indicators 323, whereby apartments falling in categories defined by the upper and lower thresholds may be differentially indicated. In some embodiments, the differential indicators or other elements of graphical user interfaces supported by the invention may utilize differential shading, color, or other differential indication method. Other input and selection features may be used with interfaces of the invention.

Throughout the figures of this document, differential indicators may be represented by shading, fill, textured elements, or other differential indication (see e.g., differential fill on differential indicators 323 of FIG. 3). Additionally, throughout the figures of this document, dates may be indicated using a month/day/year format wherein the four digits of the year are represented by asterisks (e.g., 9/5/**** in text input box 317 of FIG. 3, which represents September 5, of a particular year). The asterisks are not meant to represent wildcard characters, but are meant as placeholders for a particular year.

In some embodiments, a user 107 may toggle, click, or otherwise navigate between the different interfaces of the invention provided by system 100.

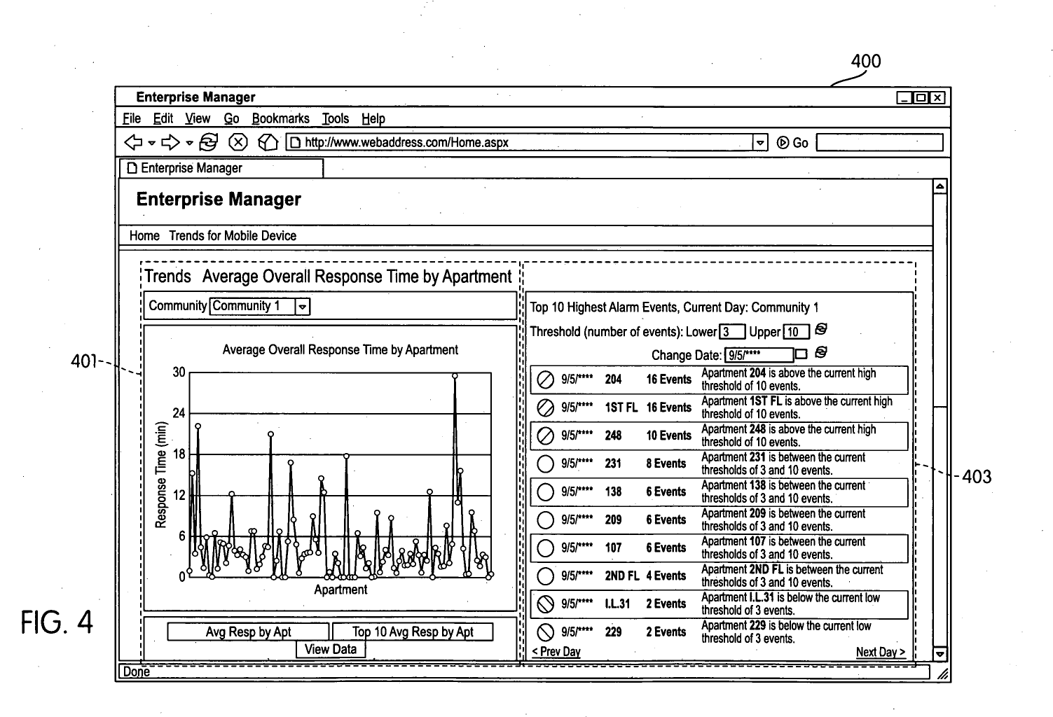

The interfaces enabled by the portal of enterprise management application 101 may include additional report types, graph types, query results, or other display of data. For example, FIG. 4 illustrates an interface 400, which is an example of a graphical user interface according to an embodiment of the invention. Interface 400 includes a section 401, which comprises a graph depicting average response times to patient distress alarm events, by apartment, for community 1. Interface 400 also includes a section 403, which comprises a table of the top 10 patient distress alarm events for a certain date, by apartment, for community 1.

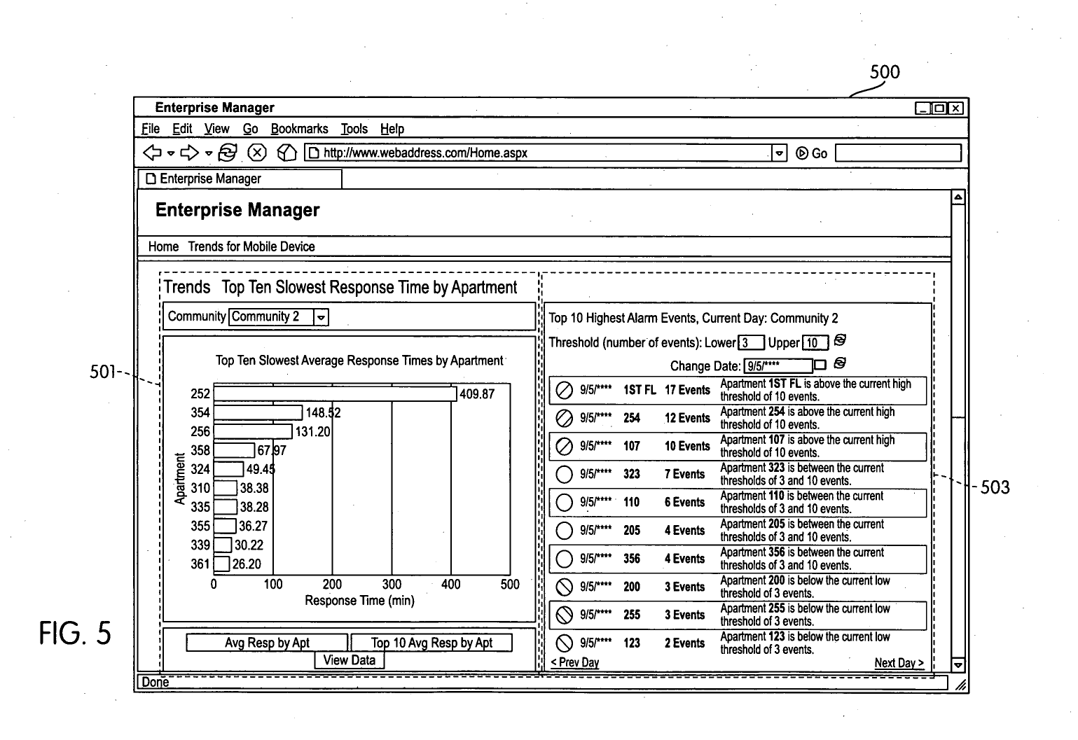

FIG. 5 illustrates an interface 500, which is an example of a graphical user interface according to an embodiment of the invention. Interface 500 includes a section 501, which comprises a graph depicting slowest average patient distress event response times by apartment for community 2. Interface 500 also includes a section 503, which comprises a table of the top 10 patient distress alarm events, by apartment, for community 2 on a certain date.

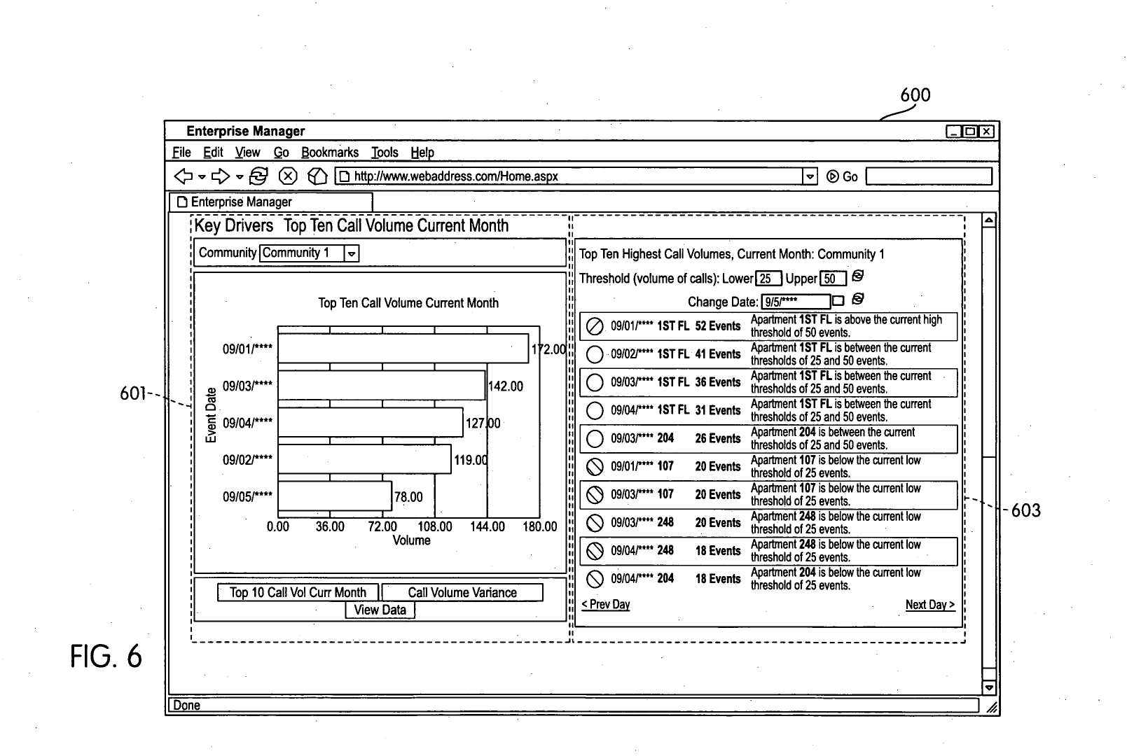

FIG. 6 illustrates an interface 600, which is an example of a graphical user interface according to an embodiment of the invention. Interface 600 includes a section 601, which comprises a graph depicting the top patient call volume days of the current month for community 1. Interface 600 also includes a section 603, which comprises a table of the top 10 patient calls, by apartment, for community 1 in a certain month. In some instances, a patient call may differ from a patient distress event. For instance, a patient call may include a call for a nurse or other medical staff member to attend to a non-urgent matter, while a distress event may include use of an “emergency button,” such as an emergency alert pendent, or other indication of an emergency situation.

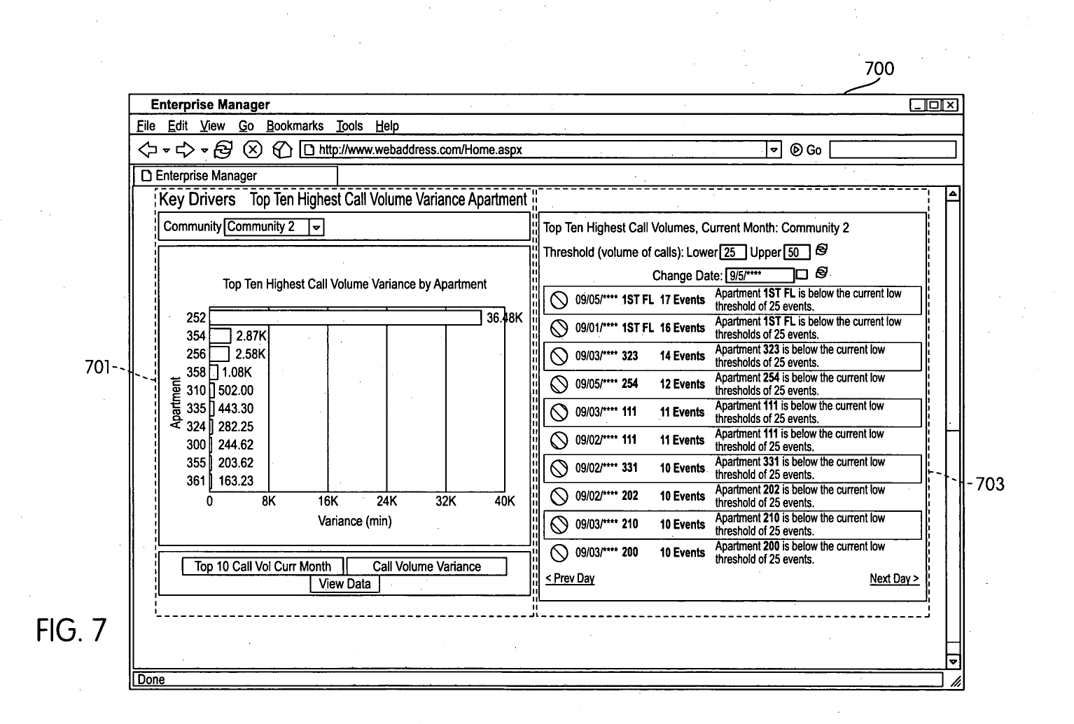

FIG. 7 illustrates an interface 700, which is an example of a graphical user interface according to an embodiment of the invention. Interface 700 includes a section 701, which comprises a graph depicting the top patient call volume variances by apartment for community 2. Interface 700 also includes a section 703, which comprises a table of the top 10 patient calls, by apartment, for community 2 in a certain month.

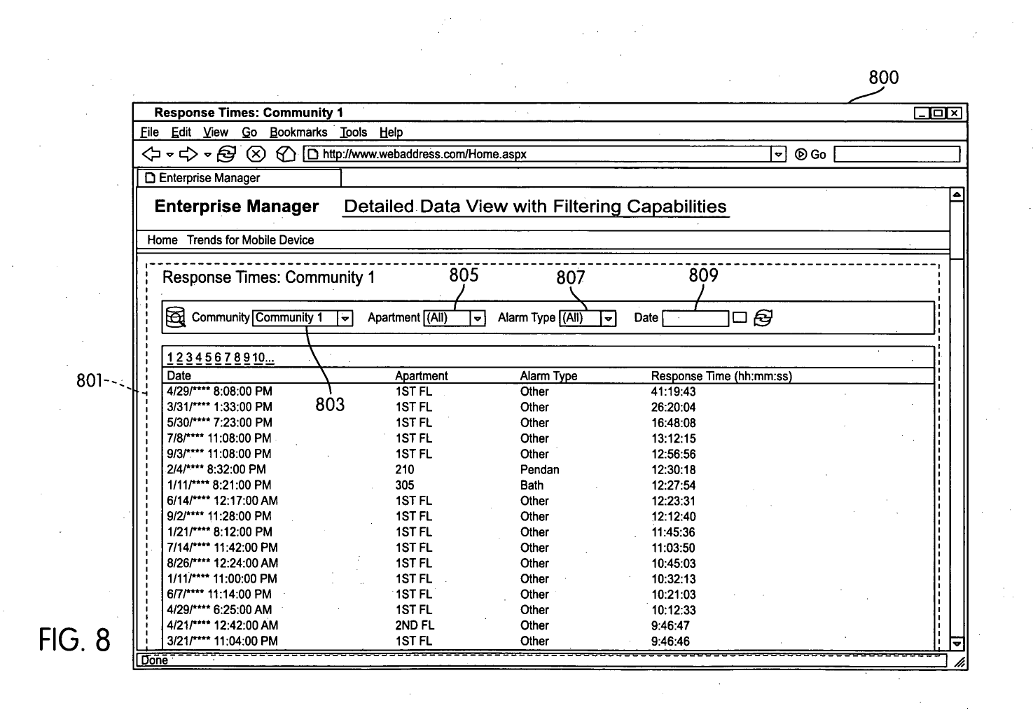

Other types of reports, query results, or data displays may also be used by the invention. FIG. 8 illustrates an interface 800, which is another example of a graphical user interface according to an embodiment of the invention. Interface 800 illustrates a section 801 which is a detailed data report with filtering capabilities that reflects patient alarm data, including: the date of an alarm, the apartment where the alarm originated, the alarm type, and the response time. Other reports displaying other data may be used. As described above, the interfaces of the invention enable various data selection and filtering capabilities wherein a user 107 may further provide report parameters or otherwise specify the content, type, and manner of data to be displayed by the interfaces of the invention. For example, a user 107 may have arrived at interface 800 by previously indicating report, query, or other parameters instructing enterprise management application 101 to display data regarding patient distress alarm response times. Once a user 107 arrives at interface 800, the user may use drop down input regions 803-809 to filter the patient alarm data by, for example, community, apartment, alarm type, and date. Other filtering parameters may also be available for use.

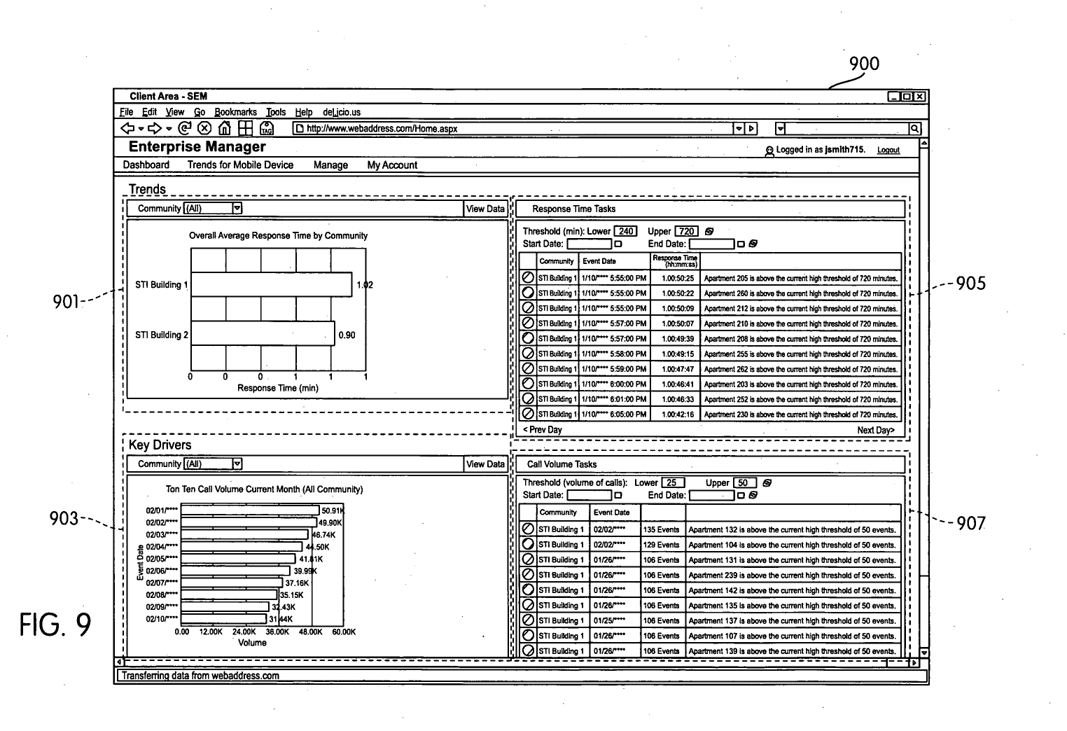

FIGS. 3-8 illustrate examples of interfaces displaying various types of reports enabled by system 100. In some embodiments, system 100 may enable a “dashboard” interface that provides a plurality of information or reports (e.g., graphs, charts, lists, or other reports). FIG. 9 illustrates an interface 900, which is an example of a graphical user interface that provides a dashboard view of data regarding assisted living facilities. Interface 900 may include a plurality of sections (e.g., sections 901, 903, 905, and 907 or other sections) that may provide reports or information regarding assisted living facilities 105 such as, for example, average patient call or event response times for various buildings in one or more communities, top patient call or event volume dates for a certain period of time (e.g., for a certain month), a listing of response times for events that exceed a certain threshold response time, a listing of apartments whose total number of calls and/or events exceed a certain threshold of events for a single day, and/or other reports or information, including any of the reports or information discussed in any of the figures herein. As such, the dashboard interface enables a user 107 to select multiple reports or information sets and view those reports or information sets on a single interface.

In some embodiments, a “dashboard” interface (e.g., interface 900) may be a homepage interface for a user 107 of system 100. For example, when a user 107 accesses the portal provided by system 100 and enters any identification and/or security information required for access (e.g., username, password, or other information), the user may be presented with a “dashboard” interface. In some embodiments, the dashboard homepage interface may include a set of “standard” reports that are presented to the user 107. In some embodiments, a user 107 may customize the type of reports included in their dashboard homepage and/or may customize the information displayed in those reports.

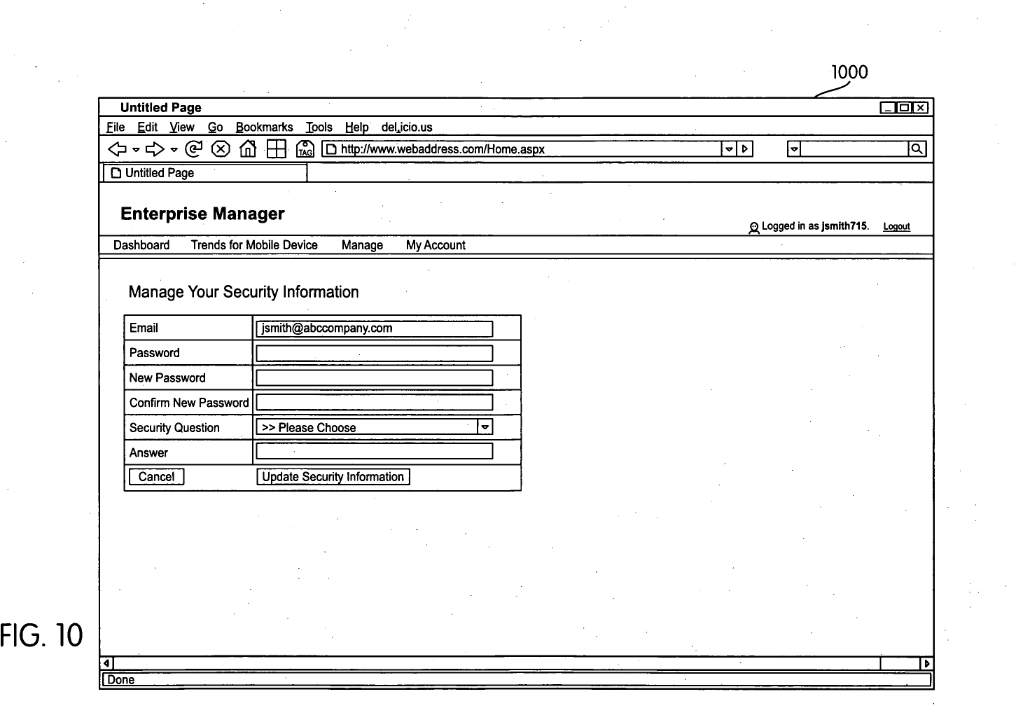

In some embodiments, system 100 may enable users 107 to manage their accounts and/or access to system 100. As disclosed herein, a user account module may enable one or more user accounts to be maintained on system 100. Each such user account may include or be associated with information relating to a specific user such as, for example, user name, password, email address, home address, position or title, phone numbers, fax numbers, permission information (e.g., for determining the level of access the user has to system 100), and/or other information. FIG. 10 illustrates an interface 1000, which is an example of a graphical user interface according to an embodiment of the invention, wherein a user 107 may manage his or her security information (e.g., password, associated email address or other security information) and/or other information associated with his or her user account. For example, interface. 1000 may include a text entry area wherein a user 107 can specify the email address associated with the user accounts. The email address associated with a user account may be used for one or more purposes such as, for example, a destination where messages, alerts, or tasks from system 100 may be sent. In some embodiments, if a user forgets his or her password, the new password may be sent to the associated email address. In some embodiments, the email address may be used as a username. The email address or other content information associated with the user account may have other uses. Interface 1000 may also include text entry areas wherein an old password may be entered, a new password may be entered and confirmed, security options relating to how the password is to recovered when lost/forgotten, an answer to a question that serves as a key to a lost/forgotten password, and/or other elements.

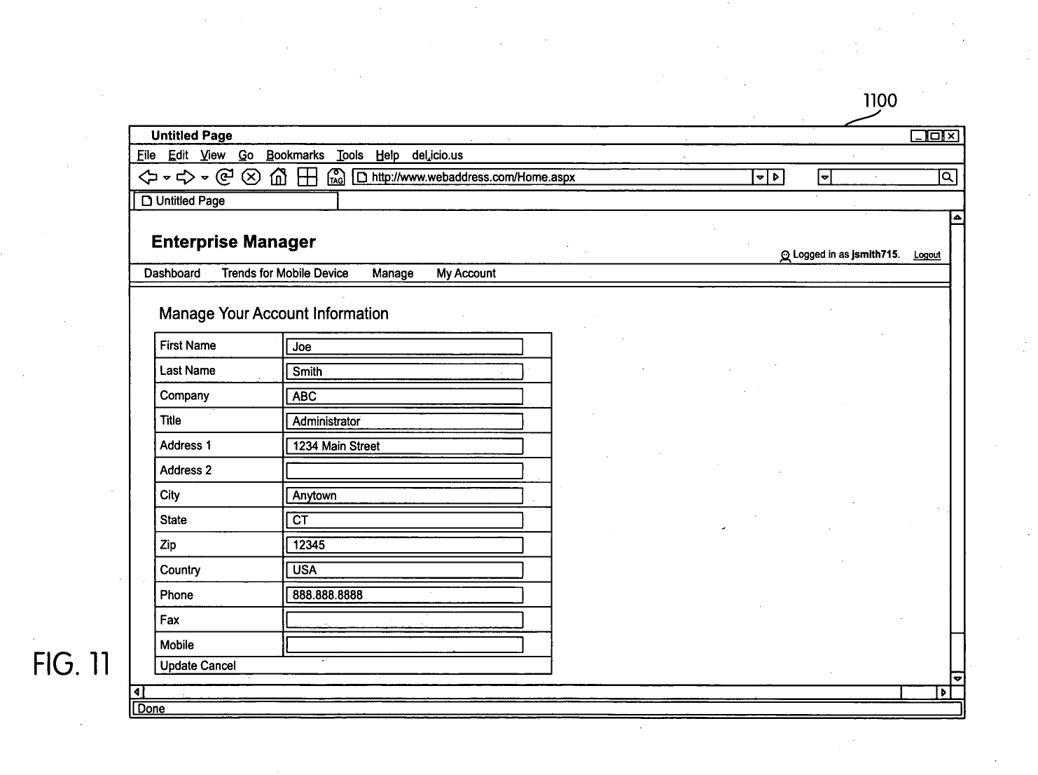

System 100 may enable other interfaces wherein users 107 may manage their user account information. FIG. 11 illustrates an interface 1100, which is an example of a graphical user interface wherein a user 107 may manage additional user account information. For example, interface 1100 may enable users to enter or edit their name, company affiliation, title, address, phone, fax or cell number (or other contact info), or other information.

System 100 may also enable certain users 107 (e.g., an upper level manager) to manage the user accounts and/or access of other users 107 (e.g., facility managers or personnel). FIG. 12 illustrates an interface 1200, which is an example of an interface wherein a first user can manage the accounts of one or more additional users. In some embodiments, interface 1200 includes a section 1201, wherein various individual user accounts may be listed and selected for editing or other management. Section 1201 illustrates that there may exist several accounts having the name of the same person associated therewith. Having multiple accounts associated with the same person illustrates how different levels of permissions may be allocated to a single person. For example, a certain user 107 may have access to one or multiple views for different buildings or sets thereof or different communities or sets thereof. These different permissions may be allocated via multiple accounts to the same person. Other permission schemes may be employed, including schemes wherein a single account includes all of the permission information for a single individual user.

In some embodiments, interface 1200 includes a section 1203, wherein individual selected user accounts can be edited. For example, use of the “is approved” checkbox illustrated in section 1203 may enable a certain account to be turned off or on. This may be especially useful in situations where permissions are controlled using multiple accounts for the same user (e.g., selectively editing turning off permissions for a first set of buildings that the user previously had permissions to, while leaving on the user's permissions to a second set of buildings). Furthermore, the “roles” checkboxes illustrated in section 1203 may enable definition of a user's role. The user's role may further aid in defining the user's permissions or ability to manage the accounts of other users. For example, if a certain user is indicated as including an “admin” role, that user may be able to edit other user's accounts by way of, for example, a “manage” tab available to the user via the portal of system 100. A “communities” area of section 1203 may enable definition of one or more communities, buildings, or other areas that a user may have permissions for/access to. An “email login info” button may enable a manager of user accounts to send a user their account login info (e.g., username, password) or other account information to the user's email address.

An account management interface, such as interface 1200, may include other features or functions.

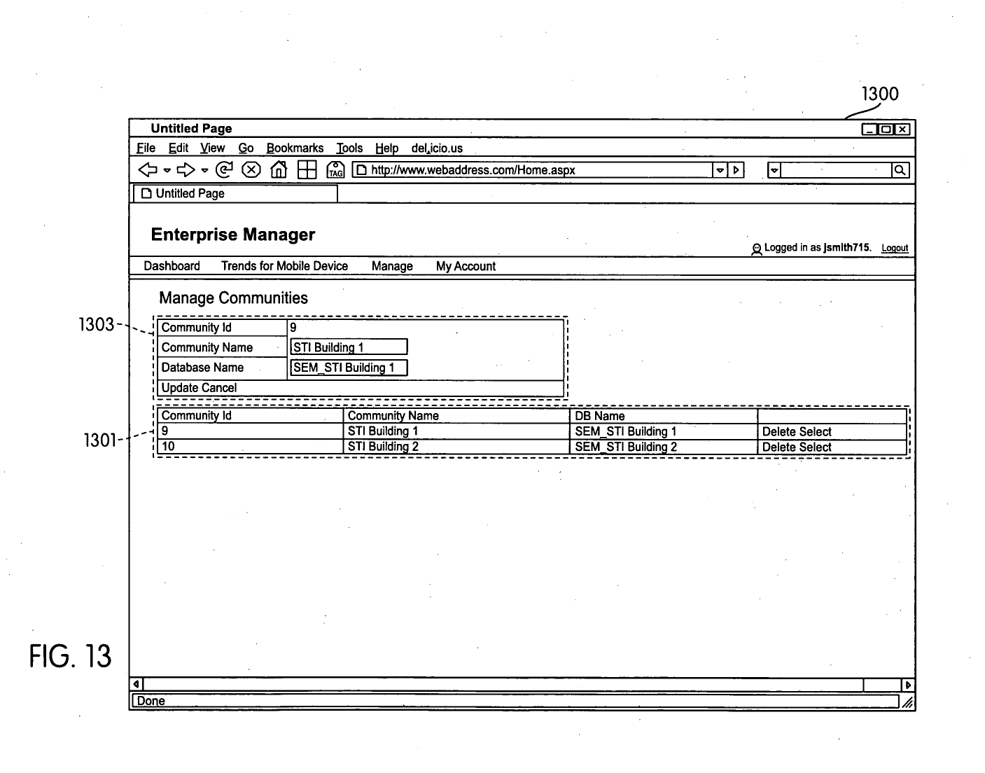

System 100 may also enable certain users 107 (e.g., upper level managers) to manage and/or view data regarding the one or more assisted living facilities 105 and/or one or more regions each having one or more assisted living facilities 105 therein. FIG. 13 illustrates an interface 1300, which is an example of an interface for viewing data regarding and managing assisted living facilities 105. In some embodiments, interface 1300 may include a section 1301, which is a list of the assisted living facilities 105 available for management. In some embodiments, a user 107 may select an assisted living facility listed in section 1301 for management. Certain data regarding the selected assisted living facility 105 may be viewed and/or edited in a section 1303. In some embodiments the information regarding with a selected assisted living facility that may be viewed and/or edited via section 1303 may include the ID number of the selected facility, the facility name, the database wherein data regarding the selected facility is stored, (e.g., information from different assisted living facilities 105 may be stored in different sections of database 103 or different individual databases comprising database 103), and/or other information. In some embodiments, the information regarding the selected assisted living facility may be edited using one or more text entry boxes provided in section 1303. In some embodiments, other information associated with assisted living facilities 105 may be viewed and/or edited in an assisted living facility management interface.

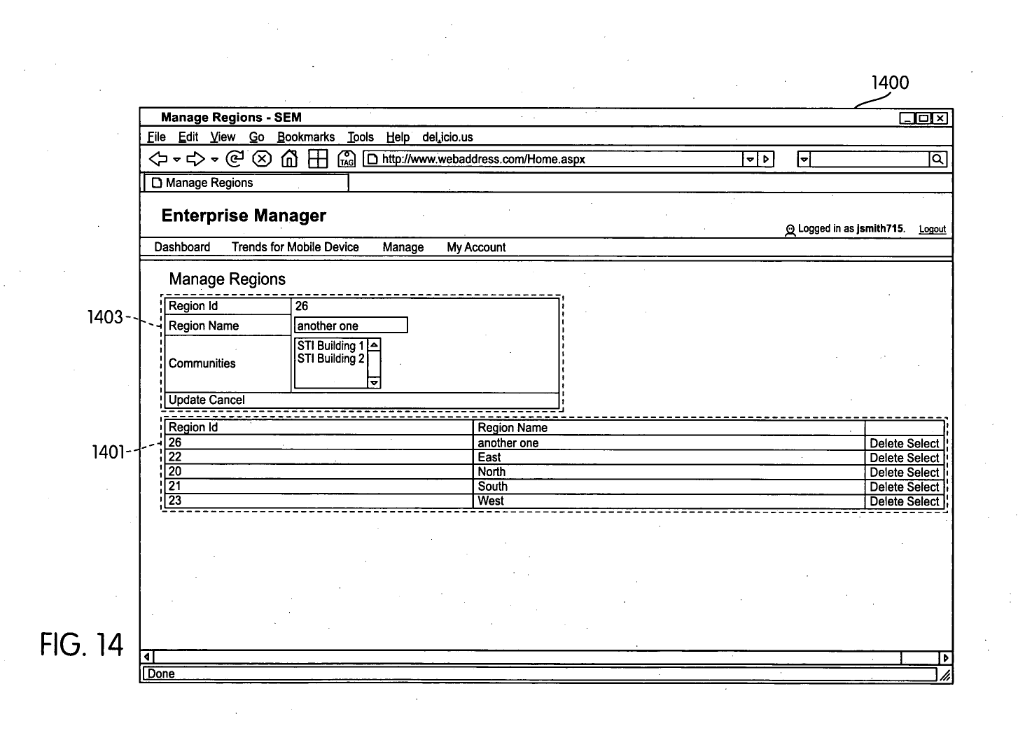

FIG. 14 illustrates an interface 1400, which is an example of an interface for viewing and/or managing assisted living facility regions, each region having one or more assisted living facilities 105 therein. In some embodiments, interface 1400 may include a section 1401, which is a list of assisted living facility regions available for management via system 100. In some embodiments, a user 107 may select an assisted living facility region from the list in section 1401 for management. Interface 1400 also includes a section 1403, wherein information associated with assisted living facility regions may be viewed and/or edited. Information associated with assisted living facility regions may include a region id number, a region name, the assisted living facilities 105 (e.g., communities) associated with the region, and/or other information. In some embodiments, interface 1400 may include one or more text entry boxes wherein edits to some or all of the information associated with assisted living facility regions may be made.

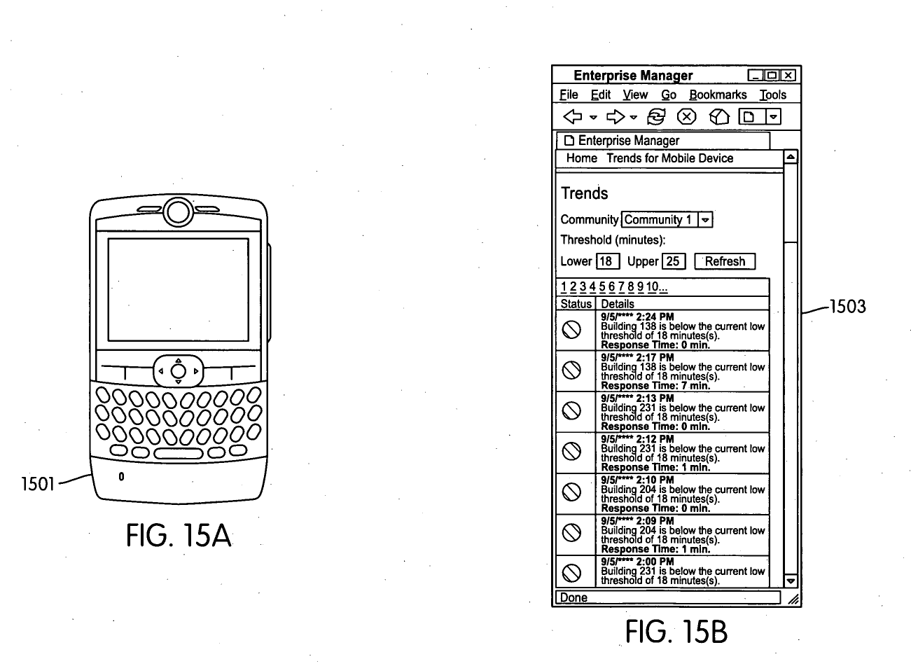

As described above, users 107 may access system 100 and database using mobile or wireless user devices. As such, one or more modules 111a-n (e.g., an interface module) of enterprise management application 101 may enable the portal of system 100 (e.g., web portal) to present graphical user interfaces compatible with mobile devices. FIG. 15A illustrates a wireless mobile user device 1501 which may display an interface of enterprise management application 101. Other mobile user devices may be used. FIG. 15B illustrates an interface 1503, which is an example of a mobile graphical user interface that may be displayed on a mobile user device.

In some embodiments, enterprise management application 101 may enable alerts to be sent to one or more users 107. For example, in some embodiments, alerts may be sent to high level business managers to keep them apprised of the operations of one or more assisted living facilities 105 or for other purposes. In another example, alerts may be sent to facility managers or health care staff on site at an assisted living 105 facility to keep them apprised of conditions at the assisted living facility, to assign tasks to certain personnel, to update tasks assigned to personnel, or for other purposes. Alerts may be sent to other users 107 for other purposes.

One or more modules 111a-n (e.g., an alert module) of enterprise management application 101 may support generating alerts for one or more users 107. Alerts may be automatically generated or generated on demand (manual generation). In some embodiments, automatically generated alerts may be sent to a specific user 107 according an alert profile associated with the specific user. In some embodiments, the alert profile may be part of an individual user's user account. For example, in some embodiments, an alert profile for a user 107 may include information as to the content of an alert (e.g., content preferences), the format of an alert (e.g., format preferences), the delivery method of an alert (e.g., delivery preferences), the timing of an alert (e.g., timing preferences), or other preferences. Content preferences may specify the specific information contained within an alert (e.g., top patient distress alarm response time by apartment for community 1). Delivery preferences may specify the format and/or delivery method by which an alert is delivered, for example, via email, phone, text message, instant message, fax, or other delivery method. Timing preferences may include a schedule or set of triggering conditions upon which alerts will be delivered (e.g., alerts sent every hour or alerts sent when average patient distress response time in community 1 exceeds a threshold). Other preferences dictating other aspects of alerts may be used.

In some embodiments, specific preferences may be tied to specific triggering conditions. For example, when a first set of triggering conditions is met, an alert may be sent according to a first set of content preferences. When a second set of triggering conditions is met, an alert may be sent according to a second set of content preferences. Other preferences (e.g., delivery or other preferences) may be tied to specific triggering conditions.

When a condition matching the triggering conditions of the alert profile of a user 107 is detected by enterprise management system 101 (e.g., a an average response time for a certain community rises above a predetermined threshold), the alert module may generate an alert according to the user's content preferences (e.g., the content preferences are used similar to report parameters, wherein a report or query module applies the content preferences against database 103 to generate the content for the alert). The alert may then be sent to the user 107 according to the user's alert profile (e.g., in the format and by delivery method specified by the delivery preferences, and/or according to other preferences).

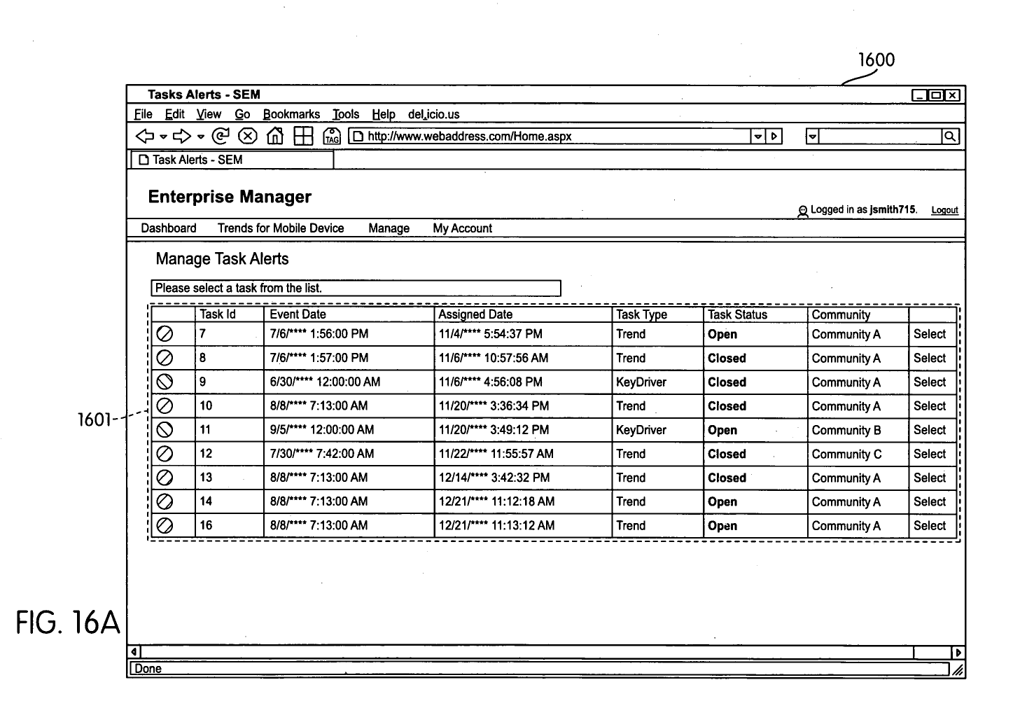

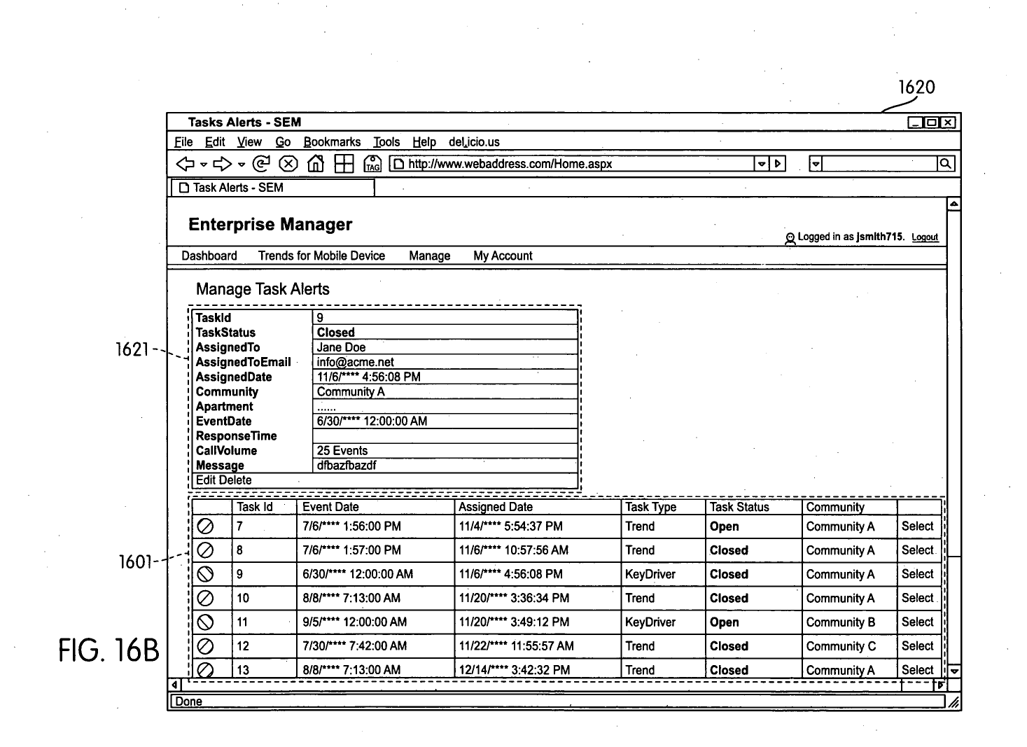

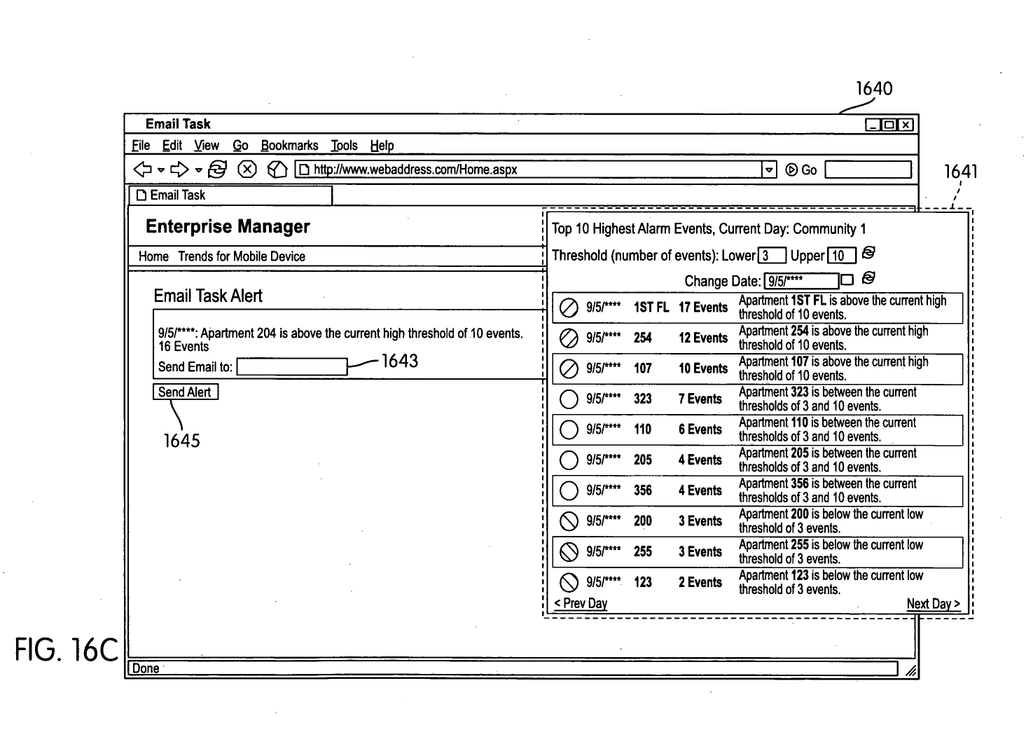

In some embodiments, system 100 (e.g., alert module of enterprise management application 101) may support creation, storage, and communication regarding one or more tasks. A task may be a type of alert and may include a set of instructions other set of information regarding one or more assisted living communities 105. For example, FIG. 16A illustrates an interface 1600, which is an example of a task alert management interface according to an embodiment of the invention, wherein task alerts or other alerts may be managed. Interface 1600 includes a section 1601, which lists various tasks that exist on system 100. Section 1601 or other task list may include information regarding each of the tasks listed such as, for example, a task ID, an event date (e.g., when the event was recorded by a monitoring device 113 of system 100—for example, when a patient has fallen and called for help), an assigned date (e.g., a date when the task was assigned or communicated to the personnel responsible for performing, attending to, responding to, overseeing, or managing completion of the task), a task type (e.g., a “trend” task type may indicate that the task is a notification of a certain trend in assisted living facility data; a “keydriver” task type may include a call volume per room task alert), task status (e.g., has the task be completed or attended to), the one or more assisted living facilities 105 (e.g., communities) associated with the task, a hyperlink or other pointer enabling a user 107 to view a listed task, edit a listed task, assign a listed task, send an alert relating to a listed task, a differential indicator of the task's importance or urgency (e.g., high alert items such as, for example, a patient distress call or indication of dangerous health conditions may be indicated with a red colored differential indicator, while lower alert indicators such as, for example, an alert that indicates that a certain community is running low on bedpans may be indicated with a yellow differential indicator), or other list features.

FIG. 16B illustrates an interface 1620, which is an example of a detailed task management interface according to an embodiment of the invention. To arrive at detailed task management interface 1621, a user 107 may, for example, select a task from a task list of a task management interface (e.g., a user may click on a hyperlink to a task listed in section 1601 of interface 1600). Interface 1620 may include a section 1621, which may provide information relating to a specific task, such as, for example, a task ID, a task status, one or more personnel assigned to the task, an email address associated with the task, a date and/or time when the task was assigned, one or more assisted living facilities 105 associated with the task, one or more apartments or specific subsections of an assisted living facility 105 associated with the task, an event date, a response time associated with the task (e.g., a time from when the task was assigned until the task is completed), a call volume (e.g., the number of patient distress calls associated with the task), a specific message, instruction, or other piece of information associated with the task (e.g., information conveying instructions or other information via text, audio, video or other format), or other information. Interface 1620 may also include a section or a link to an interface wherein a user 107 may create, edit (including editing the information presented in sections 1601, 1621, or other sections or interfaces), and/or delete one or more tasks.

The alert features disclosed above, may be utilized deliver communications regarding one or more tasks. For example, the alert features disclosed herein may be used to send an email or other communication that assigns and/or notifies appropriate personnel associated with a task. FIG. 16C illustrates an interface 1640, which is an example of an interface according to an embodiment of the invention that may be utilized to manually send alerts relating to a task to users 107. FIG. 16C also illustrates a task alert 1641 that displays the top ten high patient distress alarm events, by apartment, on a certain day at community 1. Interface 1641 includes a text input region 1643, wherein a user may specify a recipient email address for a recipient user for alert 1641. Other interfaces supported by enterprise management application 101 may enable alert 1641 to be sent via other delivery methods, to multiple recipients, or other options. Interface 1640 may also include a send button 1645, which, when actuated, may send alert 1641 to the recipient user.

Using the alert/task interfaces provided by system 100 a first user 107 may create, edit, assign, and communicate tasks to one or more second users 107. For example, a first user (e.g., a corporate manager) may be informed, through system 100 or otherwise) that a problematic condition (e.g., slow patient distress alarm response time) exists at a certain community. The first user may utilize system 100 to send a task alert to a second user (e.g., a local community manager) detailing the problematic condition and/or instructing the second manager to remedy the problematic condition. In some embodiments, this may be done using an on-demand alert. In some embodiments, this may be done using an automatically generated alert.

In some embodiments, the invention may include a computer/processor-readable medium that includes computer/processor-readable code that causes a processor, computer, or other computer system or part thereof to acquire and manipulate data associated with one or more assisted living facilities and otherwise enable monitoring and management of the one or more facilities. As such, the computer/processor readable code includes code for performing some or all of the features and functions described herein such as, for example, gathering data from monitoring devices and their associated systems, transmitting the data across a network, receiving the data, examining/processing the data, storing the data in a database, receiving data manipulation instructions (e.g., report parameters, query parameters, alert parameters, or other instructions), manipulating the data, enabling one or more interfaces for the receipt of data and/or instructions, enabling one or more interfaces for the presentation of data, enabling the production and transmittal of alerts or tasks, and/or for enabling other features or functions of the invention.

While the invention has been described with reference to the certain illustrated embodiments, the words that have been used herein are words of description, rather than words of limitation. Changes may be made, within the purview of the associated claims, without departing from the scope and spirit of the invention in its aspects. Although the invention has been described herein with reference to particular structures, acts, and materials, the invention is not to be limited to the particulars disclosed, but rather can be embodied in a wide variety of forms, some of which may be quite different from those of the disclosed embodiments, and extends to all equivalent structures, acts, and, materials, such as are within the scope of the associated claims.

What is claimed is:

1. A system for acquiring and monitoring operational data in assisted living facilities, the system comprising:
a plurality of event monitoring devices that gather data associated with at least one assisted living facility;
a database that receives, over a network, the data associated with the at least one assisted living facility from the plurality of event monitoring devices;
an enterprise management application that generates one or more reports regarding the at least one assisted living facility using the data associated with the at least one assisted living facility; and
a graphical user interface that displays the one or more reports to one or more user devices.

2. The system of claim 1, wherein the data associated with the at least one assisted living facility includes one or more of patient distress event data, patient distress event response data, employee time data, security system data, automatic entryway data, and inventory data.

3. The system of claim 1, wherein the plurality of event monitoring devices include one or more of patient medical alert necklaces, medical monitoring devices, staff event response devices, employee time entry systems, facility security systems, and automatic entryway systems.

4. The system of claim 1, wherein the network includes the Internet.

5. The system of claim 4, wherein the graphical user interface is accessible over the network via a web portal.

6. The system of claim 1, wherein the network includes a wireless telecommunications network.

7. The system of claim 1, wherein the graphical user interface is an alert sent to the one or more user devices over the network.

8. The system of claim 7, wherein the alert includes a task to be performed.

9. The system of claim 7, wherein the alert is automatically generated.

10. The system of claim 7 wherein the alert is generated on demand.

11. The system of claim 1, wherein the one or more user devices include one or more of a server, a desktop computer, a laptop computer, a dumb terminal, a handheld computer, a personal digital assistant, and a cell phone.

12. A method for acquiring and monitoring operational data in assisted living facilities, the method comprising:
receiving, over a network, data associated with at least one assisted living facility from a plurality of event monitoring devices;
storing the data associated with the at least one assisted living facility in a database;
generating one or more reports regarding the at least one assisted living facility from the stored data associated with the at least one assisted living facility;
presenting at least one of the one or more reports to one or more user devices via a graphical user interface.

13. The method of claim 12, wherein the data associated with the at least one assisted living facility includes one or more of patient distress event data, patient distress event response data, employee time data, security system data, automatic entryway data, and inventory data.

14. The method of claim 12, wherein the plurality of event monitoring devices include one or more of patient medical alert necklaces, medical monitoring devices, staff event response devices, employee time entry systems, facility security systems, and automatic entryway systems.

15. The method of claim 12, wherein the network includes the Internet.

16. The method of claim 15, wherein the graphical user interface is accessible over the network via a web portal.

17. The method of claim 1, wherein the network includes a wireless telecommunications network.

18. The method of claim 1, wherein the graphical user interface is an alert, and wherein presenting at least one of the one or more reports further comprises sending the alert to the one or more user devices over the network.

19. The method of claim 18, wherein the alert includes a task to be performed.

20. The method of claim 18, wherein the alert is automatically generated.

21. The method of claim 18, wherein the alert is generated on demand.

22. The method of claim 12, wherein the one or more user devices include one or more of a server, a desktop computer, a laptop computer, a dumb terminal, a handheld computer, a personal digital assistant, and a cell phone.

23. A processor readable medium comprising processor readable code that causes a processor to acquire and monitor operational data in assisted living facilities, the processor readable medium comprising:
receiving code that causes a processor to receive, over a network, data associated with at least one assisted living facility from a plurality of event monitoring devices;
storing code that causes a processor to store the data associated with the at least one assisted living facility in a database;
generating code that causes a processor to generate one or more reports regarding the at least one assisted living facility from the stored data associated with the at least one assisted living facility;
presenting code that causes a processor to present at least one of the one or more reports to one or more user devices via a graphical user interface.

24. The processor readable code of claim 23, wherein the data associated with the at least one assisted living facility includes one or more of patient distress event data, patient distress event response data, employee time data, security system data, automatic entryway data, and inventory data.

25. The processor readable code of claim 23, wherein the plurality of event monitoring devices include one or more of patient medical alert necklaces, medical monitoring devices, staff event response devices, employee time entry systems, facility security systems, and automatic entryway systems.

26. The processor readable code of claim 23, wherein the network includes the Internet.

27. The processor readable code of claim 26, wherein the graphical user interface is accessible over the network via a web portal.

28. The processor readable code of claim 23, wherein the network includes a wireless telecommunications network.

29. The processor readable code of claim 23, wherein the graphical user interface is an alert, and wherein the presenting code that causes a processor to present at least one of the one or more reports further comprises sending code that causes a processor to send the alert to the one or more user devices over the network.

30. The processor readable code of claim 29, wherein the alert includes a task to be performed.

31. The processor readable code of claim 29, wherein the alert is automatically generated.

32. The processor readable code of claim 29, wherein the alert is generated on demand.

33. The processor readable code of claim 23, wherein the one or more user devices include one or more of a server, a desktop computer, a laptop computer, a dumb terminal, a handheld computer, a personal digital assistant, and a cell phone.

34. A system for acquiring and monitoring operational data in assisted living facilities, comprising:
a plurality of assisted living facilities at different locations;
a plurality of patient monitors for monitoring patients at the facilities, the plurality of patient monitors each capable of generating an alert signal indicative of an event;
a central station including a processor for receiving the alert signal; and
a response switch for generating a response signal upon activation thereof, the response signal indicating that the event has been attended to,
wherein the central station processor determines a response time between the alert signal and the response signal and maintains a central database of response times at the different facilities.

35. The system of claim 34, wherein one or more of the plurality of patient monitors is each associated with a particular apartment within one of the facilities.

36. The system of claim 34, wherein one or more of the plurality of patient monitors is associated with a particular patient.

37. The system of claim 34, wherein the plurality of patient monitors include one or more of patient alert pendants, nurse call buttons, heart rate monitors, respiration monitors, blood pressure monitors, temperature monitors, and blood-gas monitors.

38. The system of claim 34, wherein the central station processor generates one or more graphical user interfaces displaying response time data using the central database of response times at the different facilities.

39. The system of claim 38, further comprising one or more user devices that access the one or more graphical user interfaces over a network.

40. The system of claim 39 wherein the network includes the Internet.

41. The system of claim 39 wherein the network includes a wireless network.

42. The system of claim 39, wherein the one or more user devices include one or more of a server, a desktop computer, a laptop computer, a dumb terminal, a handheld computer, a personal digital assistant, and a cell phone.

43. The system of claim 38, wherein at least one of the one or more graphical user interfaces includes a graph displaying average event response times for one or more of the plurality of assisted living facilities.

44. The system of claim 43, wherein the at least one of the one or more graphical user interfaces includes a user input region for selecting the assisted living facilities displayed in the graph.

45. The system of claim 38, wherein at least one of the one or more graphical user interfaces includes a table displaying total events for each of one or more patient apartments on a selected date in one of the plurality of assisted living facilities.

46. The system of claim 45, wherein the at least one of the one or more graphical user interfaces further includes a date input region for user input of the selected date.

47. The system of claim 45, wherein the at least one of the one or more graphical user interfaces further includes one or more threshold input regions for user input of one or more event thresholds.

48. The system of claim 47, wherein the one or more threshold input regions include an upper event threshold input region and a lower event threshold input region.

49. The system of claim 48, wherein apartments falling above the upper event threshold are differentially indicated from apartments not falling above the upper event indicator, wherein apartments falling below the lower event threshold are differentially indicated from apartments not falling below the lower event threshold, and wherein apartments falling between the upper event threshold and the lower event threshold are differentially indicated from apartments not falling between the upper event threshold and the lower event threshold.

50. The system of claim 45, wherein the at least one of the one or more graphical user interfaces further includes one or more hyperlinks to one or more charts displaying total events for one or more patient apartments on a different selected date in the one of the plurality of assisted living facilities.

51. The system of claim 38, wherein at least one of the one or more graphical user interfaces includes a graph displaying average overall event response times by apartment for one of the plurality of assisted living facilities.

52. The system of claim 51, wherein the at least one of the one or more graphical user interfaces includes a user input region for selecting the assisted living facility displayed in the graph.

53. The system of claim 38, wherein at least one of the one or more graphical user interfaces includes a graph displaying average event response times by apartment for one of the plurality of assisted living facilities.

54. The system of claim 53, wherein the at least one of the one or more graphical user interfaces includes a user input region for selecting the assisted living facility displayed in the graph.

55. The system of claim 38, wherein at least one of the one or more graphical user interfaces includes a graph displaying event volume by day during a predetermined period of time for one of the plurality of assisted living facilities.

56. The system of claim 55, wherein the at least one of the one or more graphical user interfaces includes a user input region for selecting the assisted living facility displayed in the graph.

57. The system of claim 38, wherein at least one of the one or more graphical user interfaces includes a graph displaying event volume variance by apartment for one of the plurality of assisted living facilities.

58. The system of claim 57, wherein the at least one of the one or more graphical user interfaces includes a user input region for selecting the assisted living facility displayed in the graph.

59. The system of claim 38, wherein at least one of the one or more graphical user interfaces includes a chart displaying events for one or more of the plurality of assisted living facilities.

60. The system of claim 59, wherein the chart displays one or more of a date of the event, an apartment associated with the event, an event type, and a response time for the event.

61. The system of claim 59, wherein the at least one of the one or more graphical user interfaces includes one or more of a facility region for user selection of the one or more of the plurality of assisted living facilities, an apartment input region for user selection of apartments with which the displayed events are associated, an alarm type input for user selection of an event type with which the displayed events are associated, and a date selection input for user selection of dates with which the displayed events are associated.

62. The system of claim 38 wherein at least one of the one or more graphical user interfaces include a graphical user interface for display on a mobile user device.

63. The system of claim 38, wherein at least one of the one or more graphical user interfaces includes a alert recipient input region for user input of an destination address for a recipient of an alert.

64. The system of claim 63, wherein the at least one of the one or more graphical user interfaces includes a send alert input region for manual user initiation of transmission of the alert to the recipient.

65. The system of claim 38, wherein at least one of the one or more graphical user interfaces include a graphical user interface that displays one or more task alerts, at least one of the task alerts including information from the central database of response times.

66. The system of claim 65, wherein the at least one of the one or more graphical user interfaces includes a section for editing information associated with the at least one of the task alerts.

67. The system of claim 66, wherein information associated with the at least one of the task alerts includes one or more of a user associated with the at least one of the task alerts and an email address of the user.
